(19) United States
(12) Patent Application Publication
(10) Pub. No.: US 2005/0013875 A1
(43) Pub. Date: Jan. 20, 2005

(54) Immunopotentiators in thermotherapy for cancer

(76) Inventors: Takeshi Kobayashi, Aichi (JP); Masashige Shinkai, Saitama (JP); Hiroyuki Honda, Aichi (JP); Kengo Ueno, Aichi (JP); Kenzo Ohtsuka, Aichi (JP)

Correspondence Address: JORDAN AND HAMBURG LLP, 122 EAST 42ND STREET, SUITE 4000, NEW YORK, NY, 10168, US

(21) Appl. No.: 10/493,665

(22) PCT Filed: Oct. 25, 2001
(86) PCT No.: PCT/JP01/09381

(51) Int. Cl. A61K 33/26; A61K 9/127; A61K 38/17

(52) U.S. Cl. 424/647; 514/12; 514/675

(57) ABSTRACT

The present invention relates to an immunostimulator in hyperthermia of cancer, which contains a heat shock protein-inducing compound such as geranylgeranyl acetone, etc. The immunostimulator of the present invention can effectively regress tumor tissue that is difficultly treated by hyperthermia alone by combining with the hyperthermia, and also, metastasis of cancer can be effectively inhibited substantially without side effect.

### TECHNICAL FIELD

The present invention relates to hyperthermia of cancer, in particular, it relates to an immunostimulator in hyperthermia using magnetic fine particles. The immunostimulator of the present invention markedly improves therapeutic effects of cancer in hyperthermia.

### BACKGROUND ART

At present, the main stream of cancer therapy is a surgical therapy. In the surgical therapy, the most significant problem is metastasis of cancer. Surgical operation is carried out by capturing tumor with naked eyes of a medical doctor oneself and it is removed. Thus, it cannot remove until the tumor becomes a certain size, but at such a stage, the cancer causes metastasis, or else, there is a possibility of not removing the tumor completely. Thus, it cannot help using an anticancer agent after the surgical operation. However, due to significant side effects, use of an anticancer agent is restricted in many cases.

As another therapeutic method of cancer, there is a hyperthermia. This is a therapeutic method of heating and killing tumor tissue by utilizing the characteristics that the tumor tissue has slightly high thermal sensitivity than a normal tissue at slightly higher temperature range (42° C. to 45° C.) than a body temperature. In the hyperthermia presently carried out, heating is carried out by irradiating a radio wave from outside the body which utilizes the slight difference in absorption of the ratio wave from that of a body tissue, so that there is a problem that a surface of the body is overheated. Thus, this is not a therapeutic method which can effectively treat a tumor at a deep portion or a small tumor alone.

The present inventors have already proposed a hyperthermia of cancer that uses magnetite with a order of submicrons as a heat generating material, as a novel therapeutic method of cancer in which the above-mentioned problems involved in the above-mentioned cancer therapy had been solved. This therapeutic method uses either magnetite cationic liposome (MCL) in which magnetite is coated with cationic phospholipid and optionally adhered by liposome that is fixed with an antibody specific to cancer cells or CMC magnetite in which magnetite is dispersed in a carboxy-methylcellulose (CMC) solution, or uses needle-shaped molded magnetite that is molded and solidified in a needle-shape.

Magnetic fine particles such as MCL, etc. specifically adsorb to cancer cells. Thus, absorption of electro-magnetic wave by tumor tissue is excellent than body tissue, and tumor tissue alone can be selectively heated, whereby hyperthermia due to inductive heat can be effectively carried out. According to this therapeutic method, it has been found that it has high antitumor effect by not only killing tumor tissue by heat, but also strengthening immune which is a prevention system in a body. In the immunostimulator, it has been considered to participate in heat shock protein (HSP).

However, even when the above-mentioned hyperthermia using magnetic fine particles such as MCL, etc. is employed, there are some cases in which therapeutic effects of cancer cannot be completely and sufficiently obtained.

Accordingly, in hyperthermia of cancer, it has been desired to obtain a manner which can markedly heighten therapeutic effects of cancer.

### DISCLOSURE OF THE INVENTION

The present invention is based on the findings that in hyperthermia of cancer, a certain kind of a compound is administered to tumor tissue, induction of heat shock protein (HSP) is progressed, and as a result, immune of a host is activated whereby an effect of hyperthermia of cancer is heightened.

That is, the present invention is an immunostimulator containing a heat shock protein-inducing compound in hyperthermia of cancer.

In the cancers in the present invention, any cancers which can be treated by hyperthermia are included, in particular, malignant brain tumor such as glioblastoma, etc., melanoma (malignant melanoma), breast cancer, prostate cancer, and the like are preferred.

In the hyperthermia of cancer according to the present invention, any therapeutic methods for treating cancer by heating tumor tissue are included, and preferably hyperthermia using magnetic fine particles. The magnetic fine particles mean fine particles having magnetism such as iron, cobalt, nickel, etc. and their compounds, etc., in particular, fine particles having magnetism such as magnetite, etc. A diameter of the magnetic fine particles is 10 to 70 nm, preferably about 40 nm.

The hyperthermia of cancer using the magnetic fine particles is a hyperthermia of cancer using magnetic fine particles as a heat generating material. In this therapeutic method, as a heat generating material, magnetic fine particles that are coated with a cationic phospholipid, or in some cases, CMC-magnetic fine particles in which a cationic liposome (CL) which is a liposome to which an antibody specific to cancer cells is fixed or magnetic fine particles are dispersed in a carboxymethylcellulose (CMC) solution may be used, or else, a needle-shaped molded product in which the magnetic fine particles are molded and solidified into a needle-shape may be used.

The cationic liposome (CL) in the present invention is a material in which magnetic fine particles such as magnetite, etc. are coated by double membranes of phospholipid containing a cationic lipid. As the phospholipid, preferred are glycerophospholipid such as phosphatidylcholine, phosphatidylethanolamine, phosphatidylserine, phosphatidylinositol, phosphatidylglycerol, diphosphatidylglycerol, phosphatidic acid, etc. and sphingolipid such as sphingomyelin, etc., that may be singly or a mixture thereof, in particular, phosphatidylcholine, phosphatidylethanolamine and phosphatidylglycerol are preferred singly or a mixture thereof. As the cationic lipid, there may be mentioned a cationic lipid with a carbon number of 10 to 18 or so, in particular, N-(α-trimethylammonioacetyl)-didodecyl-O-glutamate is preferred. A mixing ratio of the phospholipid and the cationic lipid is 10:1 to 1:1, preferably 4:1 to 3:1. A thickness of the phospholipid dual membranes is 5 to 40 nm, preferably about 10 nm.

CL may be prepared, for example, as follows. A chloroform solution of the above-mentioned mixture of the lipids is charged in an eggplant shaped flask, and chloroform is evaporated by a rotary evaporator under reduced pressure whereby the lipids mixture is evaporated to dryness inside of the flask in a film state. Next, magnetic fine particles that are previously made in colloidal state are added to the flask with a suitable amount, and the remaining material is subjected to ultrasonic wave treatment. According to this procedure, CL is prepared. A CL in which magnetite was used as the magnetic fine particles is called to as magnetite cationic liposome (MCL).

The CMC-magnetic fine particles can be produced as mentioned below. Magnetic fine particles in an amount of 0.1 to 1.5 g, preferably 0.3 g are charged in a stirring apparatus, and distilled water is added thereto until an amount thereof finally becomes 10 ml. This is warmed in a thermostat chamber at 6° C., and 10 to 30 ml, preferably 20 ml of a 6% CMC solution is added. After stirring the mixture for 30 minutes, ultrasonic wave treatment was applied for 15 minutes to prepare CMC-magnetic fine particles.

The needle-shaped molded product of the magnetic fine particles can be produced as mentioned below. Magnetic fine particles and CMC are dissolved in water in the weight ratio in the range of 4:1 to 15:1, preferably 8:1 and stirred for 90 minutes. This is molded by using an extrusion type molding machine with a diameter of 0.1 to 2.0 mm and a suitable length, preferably in comply with a size of the tumor, particularly preferably molded with a size of 80 mm, and after natural drying for 2 days, they are prepared by drying under hot wind at 80 to 100° C. to prepare the product.

Hyperthermia of cancer using MCL is carried out as follows. The thus prepared MCL as mentioned above is administered to tumor tissue by local administration, artery injection, intravenous injection, abdominal injection, etc., then, the MCL is specifically adsorbed by the tumor tissue. When a magnetic field is irradiated, the tumor tissue to which the MCL is adsorbed is superior in absorption of the electromagnetic wave to that of the body tissue, so that the tumor tissue alone is selectively heated. The tumor tissue that is temperature-sensitive is killed by an induced heat.

The hyperthermia by MCL is capable of regressing tumor (metastasis cancer) at the portion to which no MCL is administered. This shows that anti-tumor immune is induced by the hyperthermia using MCL. The immunostimulator of the present invention provides extremely excellent cancer therapeutic effect by more strongly inducing an immune-induction in the hyperthermia of cancer.

The hyperthermia using the CMC-magnetic fine particles can be carried out according to the therapeutic method using the MCL.

The hyperthermia of cancer using a needle-shaped molded product of magnetic fine particles can be practiced as mentioned below. The needle-shaped molded product of the magnetic fine particles prepared as mentioned above is applied to the tumor tissue by local injection, etc., and a magnetic field is irradiated thereto, the tumor tissue existing a needle-shaped molded product of the magnetic fine particles is superior in absorption of the electro-magnetic wave to that of the body tissue, so that the tumor tissue alone is selectively heated. The tumor tissue that is temperature-sensitive is killed by an induced heat. The hyperthermia using the needle-shaped molded product of the magnetic fine particles is also capable of regressing the tumor (metastasis cancer) at the portion to which no needle-shaped molded product is applied. This means that anti-tumor immune is induced by the hyperthermia using the needle-shaped molded product of the magnetic fine particles. The immunostimulator of the present invention provides extremely excellent cancer therapeutic effect by more strongly inducing an immune-induction in the hyperthermia of cancer

The heat shock protein (HSP) in the present invention means a protein in which synthesis thereof is induced by heat shock, and for example, it may include HSP90, HSP70, HSP60, etc., and particularly preferred is HSP70.

The heat shock protein-inducing compound in the present invention means a compound which promotes induction of a heat shock protein, and may include, for example, an isoprenoid such as geranylgeranyl acetone (GGA), retinoic acid, etc.; a lower aliphatic alcohol such as ethanol, etc.; heavy metal ion such as zinc, cadmium, etc.; arsenic; selenium; an anticancer agent such as TNF-α (tissue necrosis factor α), cisplatin, 5-FU (5′-fluoro-uracil), adriamycin, etc.; geldanamycin, herbimycinnad, etc.; lidocaine, etc. as a local anesthetic; carbonylcyanid-3-chlorophenylhydrazone, etc. as an uncoupler; lactacystin, etc. as a proteasome inhibitor, and preferred is an isoprenoid, particularly preferred is GGA.

It is uncertain about the mechanism that the immunostimulator of the present invention markedly heighten therapeutic effects of cancer in the hyperthermia, but it can be considered that in the tumor tissue to which said immunostimulator is administered, synthesis of HSP is more promoted when a thermal stress is applied by the hyperthermia. That is, it can be considered that the HSP promoted in synthesis forms a complex with a tumor antigen peptide, this complex is dissolved out from the tumor tissue, and acts as a tumor vaccine, whereby tumor-specific immune can be effectively induced, and as a result, metastasis of cancer can be controlled.

The immunostimulator of the present invention may be any form such as a liquid agent, an ointment, a solid agent, powder material, etc., and in the point of easiness of administration, etc., it is preferably a form of a liquid agent such as a solution, a suspension, an emulsion, etc.

In the immunostimulator of the present invention, depending on the form of the agent, an excipient such as water, starch, lactose, etc.; a stabilizer such as a pH controller, an antioxidant, etc.; a preservative; a dissolution aid; an emulsifier such as a surfactant, etc.; a dispersant such as sucrose, Gum Arabic, sodium citrate, etc.; a colorant; a lubricant; a binder; a disintegrator; a coating agent may be contained.

### BRIEF DESCRIPTION OF THE DRAWINGS

FIG. 1 is a photograph showing a tissue piece of tumor block after MCL administration stained by HSP. (a) is a photograph of the tissue piece when GGA was injected after administration of MCL, and (b) is a photograph of the tissue piece when no GGA was injected after administration of MCL.

FIG. 2 is a drawing showing therapeutic effects of tumor subjected to various kinds of therapy using a volume of the tumor as an index. (a) shows therapeutic effects of the case where MCL alone was administered and no magnetic field irradiation was carried out (Control Group), (b) shows therapeutic effects of the case where MCL alone was administered and magnetic field irradiation was carried out three times in total (AMF Group), (c) shows therapeutic effects of the case where MCL and GGA were administered and no magnetic field irradiation was carried out (GGA Group), and (d) shows therapeutic effects of the case where MCL and GGA are administered and magnetic field irradiation was carried out three times in total ((GGA+AMF) Group) according to the present invention.

FIG. 3 is a drawing showing a temperature at the tumor surface of rats when the magnetic field irradiation is carried out. (a) shows a temperature at the tumor surface of rats among AMF Group in which tumor was not completely regressed, and (b) shows a temperature at the tumor surface of rats of (GGA+AMF) Group.

FIG. 4 is a drawing in which change in body weights of rats of the hyperthermia (GGA+AMF) Group in which MCL and GGA are administered and three times of magnetic field irradiation are carried out is compared with those of rats of Control Group in which neither of MCL nor GGA is administered and no magnetic field irradiation was carried out.

### BEST MODE FOR CARRYING OUT THE INVENTION

An immunostimulator of the present invention is used in combination with hyperthermia of cancer. The immunostimulator of the present invention can be administered to tumor tissue prior to hyperthermia of the tumor tissue, administered to tumor tissue at the time of hyperthermia of the tumor tissue, or else, administered to tumor tissue after hyperthermia of the tumor tissue (preferably within 24 hours after the hyperthermia). In either of the cases, according to administration of the immunostimulator of the present invention, high tumor therapeutic effects can be obtained, and the immunostimulator of the present invention is preferably administered prior to the hyperthermia. Also, the immunostimulator of the present invention may be administered simultaneously with the magnetic fine particles such as MCL, etc., or may be previously administered to the whole body.

An administration method of the immunostimulator of the present invention to tumor tissue may be a local injection, an artery injection, an intravenous injection or an abdominal injection, etc., preferably by a local administration.

An administration dose of the immunostimulator of the present invention may vary depending on symptom, age, sex, etc., of the patient, and for example, it is generally 0.02 mg to 20 mg, preferably 0.1 to 0.5 mg, more preferably about 0.2 mg per 1 g of the tumor tissue.

### EXAMPLES

### Example 1

### In Vitro Experiment

(A) Cultivation of Malignant Fibrous Histiocytoma (MFH) Cells

Cultivation of MFH cells was carried out by using a 100 mm Petri dish for culturing cells into which 10 ml of a medium had been charged, at 37° C. for 24 hours in an incubator into which 5% of carbon dioxide was added. As the medium, a medium comprising Dulbecco's modified Eagle's Medium that contains 10% fetal bovine serum and, as antibiotics, penicillin G potassium (100 U/ml) and streptomycin sulfate (90 mg/ml) was used.

(B) Addition of GGA

To the MFH cells cultured for a predetermined time was directly added GGA dissolved in ethanol in a concentration of 0.1M, so that it became a concentration of 10⁻⁵ M or 10⁻⁴ M.

(C) Heating of MFH Cells

Immediately after addition of the GGA, a lid of the Petri dish for culturing the MFH cells was closely sealed with a soft plastic film, and the dish was immersed in a thermostat at 45° C. for 15 minutes to heat the contents.

(D) Measurement of HSP70

After 6 hours from completion of the heating, a culture broth was removed from the Petri dish for culturing, the cells were washed twice with a physiological saline, a liquid to dissolve the cells attached to a HSP70 immunoassay kit was added to the cells with a predetermined amount to extract HSP70 from the cells. This was quantitated by the immunoassay kit. Also, the cells extract was subjected to electrophoresis, and an amount of HSP70 was qualitatively examined by Western Blot.

(E) Results

As a result of analysis of an expressed amount of the HSP70, the expressed amount of the HSP70 after applying thermal stress was increased depending on an amount of the GGA added.

### Example 2

### Animal Experiment

(A) Experimental Individual

As experimental individuals, F344 rats (female, 7 to 8-weeks old) were used. MFH cells 1×10⁷ cultured on Petri dish for cell-cultivation were dispersed in about 50 μl of a physiologically buffered saline, and then, the cells suspension was transplanted subcutaneously to a right leg of an experimental individual by using a scalp vein needle (25G×5/8″) (Terumo Corporation) to make a cancer-carried experimental individual. Nembutal (available from Dainippon Pharmaceutical Co., Ltd.) was used for anesthesia, and that diluted to five times (50 mg/kg body weight) was administered to abdominal cavity.

(B) Preparation of MCL

A slurry of the magnetite (particle diameter 10 nm; available from Toda Kogyo Corporation) was sufficiently washed with a distilled water to remove unnecessary ion components, subjected to ultrasonic wave treatment, to obtain colloidal magnetite. 2 ml of colloidal magnetite (magnetite weight: 40 mg) was added to a phospholipid membrane which had previously been prepared at an inner wall surface of an eggplant shaped flask by using phosphatidylcholine, phosphatidylethanolamine and N-(α-trimethylammonioacetyl)-didodecyl-o-glutamate with a ratio of 2:2:1 (molar ratio; total lipid amount: 30 mg), and the membrane was swelled by subjecting to vortex stirring. The swelled membrane and the magnetic fine particles were subjected to an ultrasonic wave treatment for 15 minutes (28 W), thereafter 200 μl of 10-fold concentration of a physiological saline was added thereto, and further an ultrasonic wave treatment was carried out for 15 minutes (28 W) to obtain MCL.

(C) Administration of MCL

Under anesthesia of a rat, MCL with a concentration of 7.5 mg/ml and 3.3 mg of net weight magnetite was administered to the tumor tissue of the rat 10 days after transplantation of the MFH cells over 30 minutes by using a microsyringe pump (SP100i syringe pump, manufactured by WPI Co.), and thereafter, the rat was allowed to stand for 30 minutes. Magnetic field irradiation was carried out after 24 hours from injection of the MCL.

(D) Preparation and Administration of GGA

To 500 mg of Gum Arabic powder was adhered 1 g of GGA, 0.5% of Tween 80 solution was gradually added to the above mixture, to emulsify the mixture under stirring, and a final volume thereof was adjusted to 10 ml. This GGA suspension was diluted with a physiological saline to 100-fold, and 200 μl thereof was locally injected to the tumor tissue by using an injector after 12 hours from administration of MCL.

(E) Magnetic Field Irradiation

After 24 hours from administration of the MCL, the first magnetic field irradiation was carried out, and thereafter, further magnetic field irradiation was carried out twice with an interval of 24 hours. Accordingly, the magnetic field irradiation was carried out three times in total. The magnetic field irradiation was carried out by using an alternating magnetic field generating device (manufactured by Dai-ichi High Frequency Co., Ltd.). A high frequency magnetic field was a frequency of 120 kHz and a magnetic field strength of 384 Oe, and irradiation was carried out for 30 minutes for each time. A horizontal type coil was used for the magnetic field irradiation, and at this time, care should be taken so that a portion at which the cells were transplanted becomes a center of the coil.

(F) Measurement of Temperature at Tumor Surface

During magnetic field irradiation, a temperature at the tumor surface was measured with a lapse of time. A top end of an optical fiber thermometer was fixed on the surface of the tumor surface with a tape. As a control, a temperature at the rectum was simultaneously measured.

(G) Stain of HSP70

After 2 hours from administration of GGA, the tumor block was cut out, the tissue was fixed with 10% formalin, and after paraffin embedding, a cut piece was cut out, subjected to a paraffin removing treatment, and immunostained by using an anti-HSP70 antibody.

(H) Measurement Method of Tumor Tissue Volume

Under anesthesia of experimental individuals, a longer diameter and a shorter diameter of the tumor tissue were measured by using calipers and calculated out from the following formula.
Tumor tissue volume=(longer diameter)×(shorter diameter) ²×0.5
(I) Results

As shown in FIG. 1, as a result of HSP70 stain, in the tumor tissue to which the GGA had been administered, induction of HSP70 was confirmed.

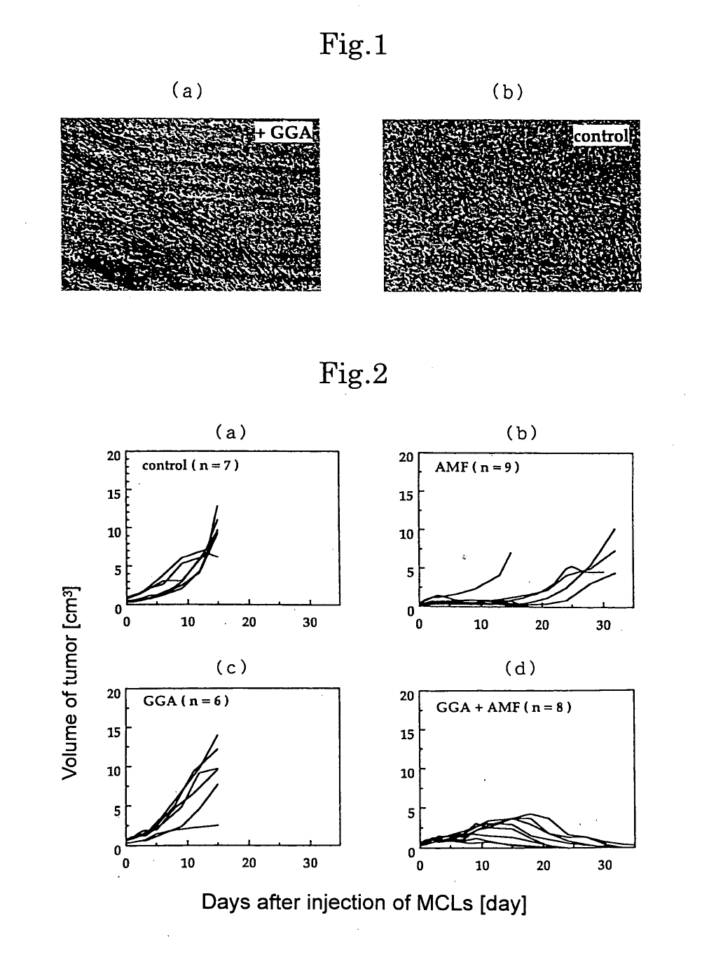

As can be clearly seen from FIG. 2, in the Group of hyperthermia alone using MCL (AMF Group), among 9 individuals, tumors of 4 individuals were completely regressed and cured, and in the Group of hyperthermia using MCL and administration of GGA in combination (GGA+AMF), tumors of 8 individuals among 8 individuals were completely regressed and cured.

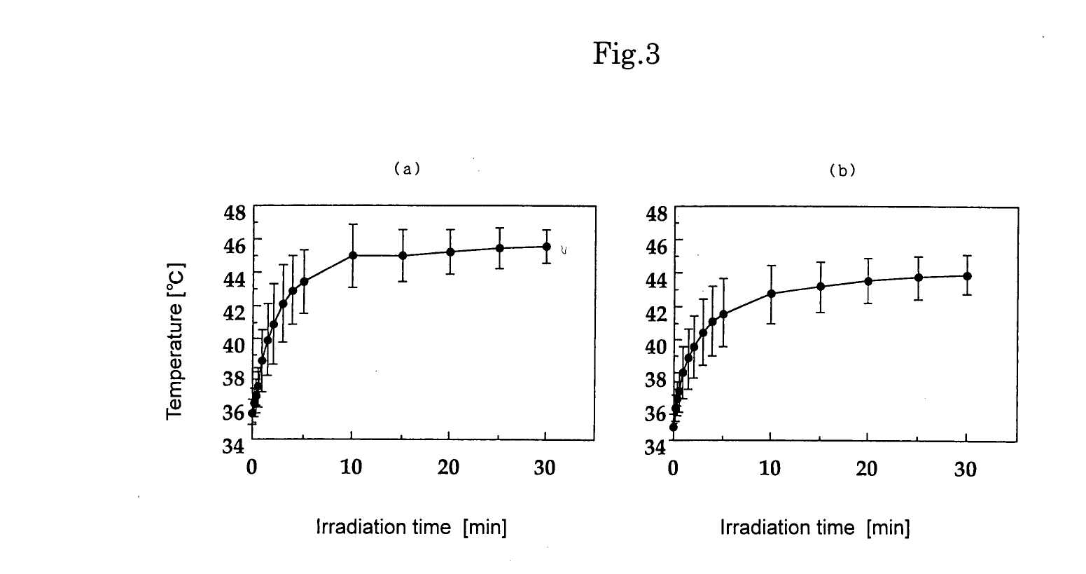

As far as the temperature at the tumor surface of rats during hyperthermia in which the cancer was not cured by the hyperthermia (AMF Group) using MCL alone is observed, there is no problem in the hyperthermia itself (see FIG. 3).

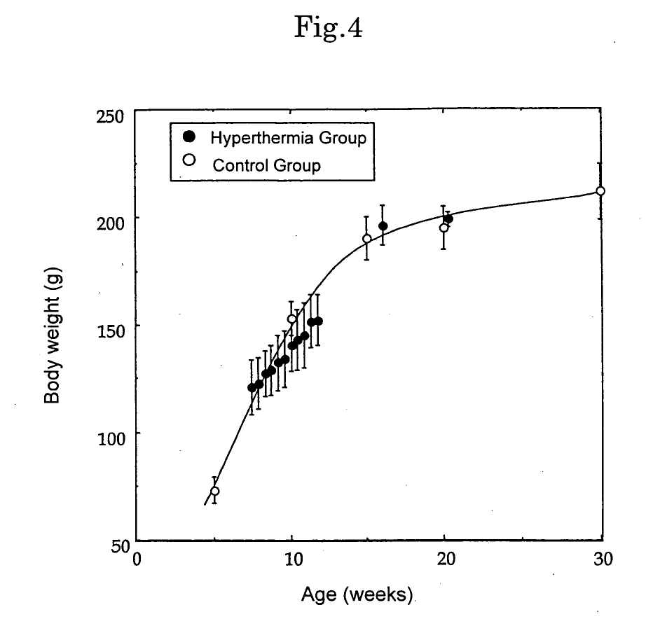

Also, as can be seen from FIG. 4, a body weight change in rats of the hyperthermia (GGA+AMF) Group in which MCL and GGA are administered and three times of the magnetic field irradiations were carried out according to the present invention is substantially the same as those of the rats in Control Group in which neither MCL nor GGA was administered and no magnetic field irradiation was carried out, so that it can be understand that a side effect of the present invention is extremely little.

(Utilizability in Industry)

When the hyperthermia of cancer was carried out by using the immunostimulator of the present invention, a tumor tissue which is difficultly treated by the hyperthermia alone, and simultaneously metastasis of cancer can be also effectively inhibited.

Moreover, according to the present invention, therapy of cancer can be carried out while markedly controlling side effects such as decrease in a body weight, etc.

1. An immunostimulator in hyperthermia of cancer which comprises a heat shock protein-inducing compound.

2. The immunostimulator according to claim 1, wherein the heat shock protein-inducing compound is selected from the group consisting of isoprenoids, lower fatty alcohols, heavy metal ions, arsenic, selenium, anticancer agents, heavy metal ions, arsenic, selenium, anticancer agents, local anesthetics, uncouplers and proteasome inhibitors.

3. The immunostimulator according to claim 2, wherein the isoprenoid is geranylgeranyl acetone.

4. The immunostimulator according to claim 1, wherein the hyperthermia of cancer uses magnetic fine particles or their needle-shaped products.

5. The immunostimulator according to claim 4, wherein the magnetic fine particles comprise magnetic cationic liposome.

6. (Canceled)

7. A tumor treating agent in hyperthermia which comprises the immunostimulator according to claim 1 and magnetic fine particles or their needle-shaped molded products.

8. The tumor treating agent according to claim 7, wherein the magnetic fine particles comprise magnetic cationic liposome.

9. The immunostimulator according to claim 2, wherein the hyperthermia of cancer uses magnetic fine particles or their needle-shaped molded products.

10. The immunostimulator according to claim 3, wherein the hyperthermia of cancer uses magnetic fine particles or their needle-shaped molded products.

11. The immunostimulator according to claim 9, wherein the magnetic fine particles comprise cationic liposome.

12. The immunostimulator according to claim 10, wherein the magnetic fine particles comprise cationic liposome.

13. A tumor treating agent in hyperthermia which comprises the immunostimulator according to claim 2 and magnetic fine particles or their needle-shaped molded products.

14. A tumor treating agent in hyperthermia which comprises the immunostimulator according to claim 3 and magnetic fine particles or their needle-shaped molded products.

15. The tumor treating agent according to claim 13, wherein the magnetic fine particles comprise magnetic cationic liposome.

16. The tumor treating agent according to claim 14, wherein the magnetic fine particles comprise magnetic cationic liposome.

17. A method of treating cancer, comprising administering a heat shock protein-inducing compound to the cancer and thereafter applying hyperthermia to the cancer.

18. A method according to claim 17, wherein the heat shock protein-inducing compound is selected from the group consisting of isoprenoids, lower fatty alcohols, heavy metal ions, arsenic, selenium, anticancer agents, local anesthetics, uncouplers and protease inhibitors.

19. A method according to claim 18, wherein the isoprenoid is geranylgeranyl acetone.

20. A method according to claim 17, further comprising, prior to said applying of hyperthermia, administering to the cancer magnetic fine particles or needle-shaped bodies molded from said magnetic fine particles.

21. A method according to claim 20, wherein the magnetic fine particles comprise magnetic cationic liposome.
